(19) United States
(12) Patent Application Publication
(10) Pub. No.: US 2026/0294250 A1
(43) Pub. Date: Oct. 1, 2026

(54) SCANNING DEVICE

(71) Applicant: ZONAPIES SL, Fuenlabrada, Madrid (ES)

(72) Inventor: Raul TRUJILLO ACEITUNO, Fuenlabrada (ES)

(73) Assignee: ZONAPIES SL, Fuenlabrada, Madrid (ES)

(21) Appl. No.: 19/481,118

(22) PCT Filed: May 2, 2024
(86) PCT No.: PCT/EP2024/062086

§ 371 Date: Nov. 3, 2025

(30) Foreign Application Priority Data
May 16, 2023 (EP) 23382459.8

(51) Int. Cl. A61B 5/00; A61B 5/107

(52) U.S. Cl. CPC A61B 5/0064; A61B 5/1074; A61B 5/1079

(57) ABSTRACT

The present disclosure relates to scanning devices for imaging human extremities. The scanning devices comprise a substantially transparent base comprising a first side and a second side, the first side being configured to accommodate the human extremity. The devices further comprise a stereoscopic camera comprising one or more infrared lasers configured to emit a laser beam which hits the second side of the transparent base in one or more incidence points. Further, one or more opaque items are located between the one or more laser beams and the one or more incidence points

The present application claims the benefit of EP 23382459.8 filed on May 16, 2023, 2023.

The present disclosure relates to devices for imaging human extremities, particularly feet. The present disclosure further relates to methods for imaging human extremities using such devices.

### BACKGROUND

Orthotic insoles are customized to patients with the objective of treating or correcting various diseases or conditions. In order to obtain a customized insole, the morphological parameters of the plantar footprint of the patient may need to be obtained.

Phenolic foams or plaster moulds have been commonly used to obtain moulds of the footprints of patients. However, these techniques require expertise to obtain a mould with the correct parameters, and in addition, they cannot directly provide a digitalized image of the plantar footprint, which can be very useful e.g. to store or manipulate the obtained data.

Different techniques which enable digitalization of data are becoming more and more popular. One way of digitalizing the morphological parameters of the plantar footprint of a patient is via digital scanners which capture the three-dimensional shape of the plantar appearance of human feet e.g. for the purpose of designing customized insoles.

These scanners generally comprise a frame comprising a transparent area and an inner enclosure which comprises three-dimensional imaging means. The transparent area is where a patient would be asked to place a foot, such that a digital image can be obtained.

With these scanners, the morphological parameters of the feet of a patient can be provided directly to a computer. A medical professional can visualize the images and parameters on a suitable display and study them in a comfortable and efficient manner. In addition, programs have been developed in order to modify such parameters and e.g., obtain personalized insoles, which can then be printed and used to treat certain conditions of the patient.

Stereoscopic cameras, also known as three-dimensional (3D) cameras, are able to capture three-dimensional images. These images can then be digitalized and further processed in a computer program.

Stereoscopic cameras can be placed inside the frame of a foot scanner and be used to obtain images of the foot sole. One problem that has been encountered with these cameras is that they use infrared lasers as depth sensors, which cause reflections during the scanning process when the lasers reach the transparent area of the scanner. These reflections lead to inaccurate or defective images of the foot sole.

In order to solve this problem, it is known to use imaging systems using infrared lasers that are not in a fixed position inside the frame. Rather they are installed on moving platforms or chassis which allow them to slide e.g. in a longitudinal direction of the scanner while taking the images. Because multiple images are obtained, the inaccuracies in individual images caused by the infrared lasers can be compensated such that the infrared lasers do not interfere in the quality of the images.

As a result, the scanning process is relatively complex and the cost of three-dimensional imaging scanners tends to be relatively high.

Three-dimensional scanners have been developed which instead of having a transparent area, have silicone membranes, where the foot soles of the patients are accommodated. Since they do not comprise a transparent base, these scanners do not have the problem of reflections. However, with these scanners, the membrane rather than the foot sole, is scanned.

The present disclosure intends to solve at least some of the above-mentioned difficulties.

### SUMMARY

In a first aspect, a scanning device for imaging a human extremity is provided. The scanning device comprises a substantially transparent base comprising a first side and a second side, the first side being configured to accommodate the human extremity. The scanning device also comprises a stereoscopic camera comprising one or more infrared lasers which are configured to emit a laser beam which hits the second side of the substantially transparent base in one or more incidence points. One or more opaque items are located between the one or more laser beams and the one or more incidence points.

The opaque items prevent the laser beams from directly hitting the second side of the substantially transparent base. When the laser beams hit the substantially transparent base, the laser beam is distorted and the scanning device obtains three-dimensional images with undesired reflections. The one or more opaque items avoid the distortion of the laser beams and therefore enable the obtention of 3D images of morphological parameters of a human extremity of good quality. At the same time, standard, well-known stereoscopic cameras without further modifications may be used, thus simplifying the design and constructions.

In some examples, the stereoscopic camera may be a static camera. The obtention of 3D images without undesired reflections may be achieved without the need of using systems which move the camera. The scanning system may be manufactured in a simpler way.

In some examples, the human extremity may be a human foot. In some examples, the substantially transparent base of the scanning device may be configured to accommodate a sole of the human foot.

Throughout the present disclosure, an opaque item may be regarded as any element which may be able to prevent the laser beams of the infrared lasers to traverse it.

In some examples, the number of opaque items comprised in the substantially transparent base may be the same as the number of infrared lasers comprised in the stereoscopic camera. In some examples, the opaque items may cover at least the area of the incidence point of the laser beams.

In some examples, the opaque items may be attached to the second side of the substantially transparent base. In some examples, the opaque items may be adhesively attached, specifically in some examples, the opaque items may be stickers.

In a further aspect, a method for imaging a human extremity is provided. The method comprises providing the scanning device for imaging a human extremity. The method comprises accommodating the human extremity on the first side of the substantially transparent base. The method further comprises scanning and obtaining a 3D image.

In some examples, processing the 3D image may comprise removing the opaque items from the obtained image. In some examples, the method may further comprise processing the 3D image and obtaining a 3D image comprising only the accommodated human extremity.

### BRIEF DESCRIPTION OF THE DRAWINGS

Non-limiting examples of the present disclosure will be described in the following, with reference to the appended figures, in which:

FIG. 1 shows a scanning device according to an example of the present disclosure;

FIG. 2 shows a cross sectional view of a scanning device according to an example of the present disclosure; and

FIG. 3 is a flow chart of a method for imagining a human extremity.

The figures refer to example implementations and may only be used as an aid for understanding the claimed subject matter, not for limiting it in any sense.

### DETAILED DESCRIPTION OF EXAMPLES

In these figures, the same reference signs have been used to designate matching elements.

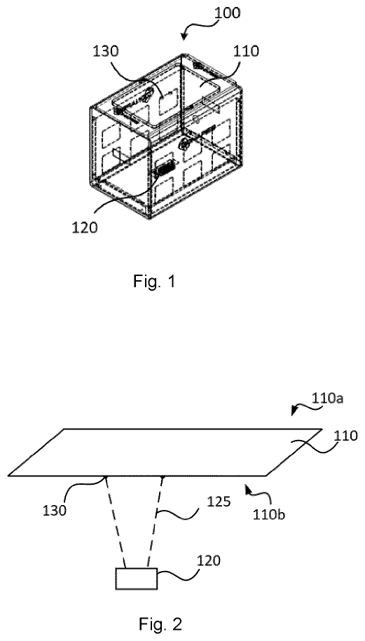

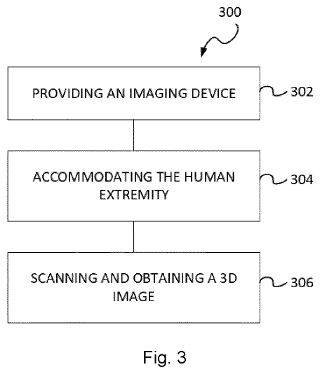

FIG. 1 schematically shows a scanning device 100 for imaging a human extremity according to an example of the present disclosure.

The scanning device 100 comprises a substantially transparent base 110 comprising a first side and a second side, the first side being configured to accommodate the human extremity. The scanning device also comprises a stereoscopic camera 120 comprising one or more infrared lasers configured to emit a laser beam which hits the second side of the transparent base in one or more incidence points. One or more opaque items 130 are located between the one or more laser beams and the one or more incidence points.

The opaque items prevent the infrared laser beams from hitting the substantially transparent base, avoiding the presence of undesirable reflections in a three-dimensional (3D) image obtained by the stereoscopic camera. The laser beams do not reach the transparent base and are not distorted, allowing the obtention of a clear 3D image of morphological parameters of a human extremity.

Therefore, the one or more opaque items located between the one or more laser beams and the one or more incidence points enable obtaining 3D images which are free from reflections and effectively scanning a human extremity accommodated on a substantially transparent base with a stereoscopic camera which does not have to be moved to obtain multiple images.

As shown in FIG. 1, the scanning device may comprise a frame. In some examples, the frame may comprise six walls which may define a polyhedron. The six walls of the frame may define an enclosure.

The upper wall of the frame may be a substantially transparent base 110. A substantially transparent base may be understood as a base which allows light to pass through it, so that objects can be clearly seen through it. The substantially transparent base 110 comprises a first side and a second side, the first side being configured to accommodate the human extremity. The first side of the substantially transparent base may be an outer base of the frame. The second side of the substantially transparent base may be an inner base of the frame, and therefore an inner side of the scanning device.

As shown in FIG. 1, the substantially transparent base 110 may comprise a substantially rectangular shape. In some examples, the transparent base may be substantially flat. In other examples, the substantially transparent base may comprise any shape which may be suitable for accommodating a human extremity.

In some examples, the scanning device 100 may be used for imaging a human foot. In some examples, the substantially transparent base of the scanning device may be configured to accommodate a sole of a human foot. The substantially transparent base 110 may be made of a material which may be able to withstand the weight of an adult human.

In some examples, the substantially transparent base 110 may be made of plastic, such as a methacrylate, or glass. Any material that enables directly imaging the extremity accommodated on the base with the stereoscopic camera 120 might be used.

Within the enclosure of the scanning device 100, a stereoscopic camera 120 may be located. In some examples, the stereoscopic camera 120 may be the camera Intel® RealSense™ D435. Experiments have been performed using this camera and have shown that image quality can be improved with the relatively simple measures presented herein. It will be clear that in other examples, other stereoscopic cameras may be used.

The stereoscopic camera 120 may comprise two or more lenses. In some examples, the distance between the lenses may be of e.g. 5-6 cm. At least part of the substantially transparent base 110 of the scanning device may be within the field of view of the stereoscopic camera 120. The field of view of the camera 120 may comprise the area across which the stereoscopic camera 120 can image i.e. the area which is visible through the camera.

In addition, the stereoscopic camera 120 comprises one or more infrared lasers. In some examples, the stereoscopic camera 120 may comprise at least one infrared laser. In other examples, the stereoscopic camera 120 may comprise one infrared laser per lens. In some examples, the one or more infrared lasers may be part of a depth sensor of the stereoscopic camera. The information derived from the infrared laser(s), in combination with the two lenses, may thus be used to obtain three-dimensional images.

The one or more infrared lasers of the stereoscopic camera 120 may generate one or more laser beams which are configured to hit the substantially transparent base 110 in one or more incidence points. The one or more incidence points may be located on the inner side of the transparent base and the one or more laser beams may be configured to hit the second side of the transparent base.

In the shown example, the static stereoscopic camera 120 is located underneath the transparent base 110 e.g. 30-40 cm away from the transparent base, such that the one or more lenses of the camera may point towards the transparent base 110. The camera may be located within the frame of the scanning device 100 in a fixed and permanent position i.e. the field of view of the camera is always the same. In some examples, the camera may be fixed to the bottom wall of the frame e.g. by mechanical means.

The stereoscopic camera may obtain 3D images which may be sent to a digital system e.g. a computer system. The camera may have a wired or wireless connection with a suitable computer system. In some examples, the computer system may be a general purpose computer, e.g. laptop or desktop, which has a suitable computer program installed on it.

In some examples, the field of view of the camera may include at least part of the substantially transparent base, one or more walls of the frame and the one or more opaque items of the substantially transparent base. The camera can obtain a 3D image of a human extremity accommodated on a part of the transparent base which may be included within the field of view of the camera. The images that are obtained may comprise a foot sole of a patient, although in other examples e.g. a hand may be imaged. In particular, the morphological parameters of the foot sole may be obtained in preferred examples.

As schematically illustrated in FIG. 1, the second side of the substantially transparent base 110 comprises one or more opaque items 130. The opaque items 130 are located between the one or more laser beams and the one or more incidence points.

The incidence points of the one or more laser beams may be on the one or more opaque items 130 instead of on the transparent base 110 i.e. the laser beams of the infrared lasers may hit the one or more opaque items instead of the second side of the transparent base.

The opaque items 130 may be made from any opaque material. In some examples, the material of the opaque items may be substantially lightweight such that they may not add an extra weight to the scanning system. In some examples, the opaque items 130 may be attached to the second side of the transparent base 110 e.g. via an adhesive. In some examples, the opaque items 130 may be stickers. Opaque stickers may be lightweight and may be easily attached to the transparent base. In other examples, the opaque items may be fixedly attached to the second side of the transparent base 110.

In some examples, the opaque items 130 may comprise an area which may at least cover the area of the incidence point of the laser beams. In some examples, the opaque items 103 may substantially have the same shape and size as the incidence points. In some examples, the opaque items 130 may have a substantially round or squared shape. In some examples, the opaque items 130 may be located at least 2 cm away from each other.

In some examples, the number of opaque items 130 comprised in the transparent base 110 may be the same as the number of infrared lasers comprised in the stereoscopic camera 120.

The field of view of the camera may include a central part of the transparent base 110 and the lenses of the stereoscopic camera 120 may be pointed towards it. The opaque items 130 may be located substantially in a central part of the transparent base 110 such that each of the one or more laser beams of the infrared lasers hits one opaque item 130.

In a further aspect of the present disclosure, a method 300 for imaging a human extremity is provided. The method comprises, at step 302, providing an imaging device as the one disclosed throughout the present disclosure. The method comprises, at step 304, accommodating the human extremity on the first side of the transparent base. The method further comprises, at step 306, scanning and obtaining a 3D image.

The method allows obtaining a clear 3D image of an extremity accommodated on the transparent base which does not comprise undesired reflections due to the distortion of the infrared lasers.

At step 302, a scanning device for imaging a human extremity is provided. The scanning device 100 may be a device as illustrated in FIG. 1.

The method further comprises, at step 304, accommodating a human extremity on the first side 110a of the substantially transparent base.

In some examples, the human extremity may be accommodated on a part of the first side 110a of the substantially transparent base which is within the field of view of the stereoscopic camera 120. In some examples, accommodating the human extremity on the first side 110a of the substantially transparent base comprises covering the one or more opaque items 130 with the extremity.

In some examples, the method may be used for imaging a sole of a human foot. A patient may accommodate their foot sole on the transparent base 110 of the scanning device. The length and the width of the foot sole of the patient may be within the field of view of the camera. The scanning device may be used for imaging a human foot of a patient who is standing and/or who is sitting.

At step 306, the method comprises scanning and obtaining a 3D image.

FIG. 2 schematically shows an example of a cross sectional view parallel to a vertical direction of the scanning device shown in FIG. 1 during a scanning process.

In some examples, scanning may comprise activating the stereoscopic camera 120 e.g. wirelessly or via an activator. The field of view of the stereoscopic camera 120 may comprise the substantially transparent base 110, such that when a human extremity may be accommodated on the first side 110a of the substantially transparent base, it is within the field of view of the camera.

As schematically illustrated in FIG. 2, when the stereoscopic camera 120 is activated, infrared laser beams 125 may be directed towards the second side 110b of the substantially transparent base. FIG. 2 also shows the substantially transparent base 110 comprising on a second side 110b two opaque items 130.

The opaque items 130 are located between the two laser beams 125 and the transparent base such that the laser beams 125 of the infrared lasers strike the opaque items 130.

After scanning, a 3D image of everything that is included within the field of view of the stereoscopic camera may be obtained.

The stereoscopic camera may provide a 3D image which may be obtained in a digital system e.g. a computer system. In some examples, the field of view of the stereoscopic camera of the scanning device 100 may include part of the transparent base 110, the opaque items 130 and one or more walls of the frame of the scanning device.

Accordingly, the three-dimensional image obtained through this method may comprise the human extremity e.g. a foot sole, the one or more opaque items on the transparent area and one or more walls of the frame of the scanning device.

In some examples, the method may further comprise processing the 3D image and obtaining a 3D image comprising only the accommodated human extremity. In some examples processing the 3D image may comprise removing substantially all details that may appear in the image except for the details concerning the human extremity. This may be done automatically by a digital system e.g. by a software. Particularly, the opaque items may be removed from the images, and the actual part of the foot sole that is “hidden” behind the opaque items may be reconstructed from the images obtained with the camera employing multiple lenses.

In some examples, the distance between the camera and the opaque items may be used to identify the exact position of the opaque items in the transparent area and remove their presence from the 3D image. Further, during the processing step, the distance between the camera and the walls of the frame may be used to identify the walls in the obtained image and successfully remove them from the initial 3D image.

In some examples, a 3D image of a foot sole of a patient may be used to design a personalized orthotic insole.

In these examples, the stereoscopic camera may further be used to obtain an image of the foot sole i.e. a two-dimensional (2D) image. A digital system may scale the image and add it to the previously obtained three-dimensional image of the foot sole.

The three-dimensional image with the 2D image may be obtained in the digital system and may further be used to study the morphological parameters of the plantar footprint of the patient. In addition, the digital system may enable drawing on the digital image e.g. indications or the location of painful spots, which may be very useful for a medical professional.

Further, the digital system may be configured to process the morphological parameters of the plantar footprint and to automatically obtain the points (A-Z) which indicate the separation between the retro capital area and the sub capital area, as well as the line formed by such points. The digital system may also allow the modification of these points e.g. the points may be moved forward or backward. In addition, the digital system may also be used to generate the divisions of the metatarsal heads which, similarly to the points (A-Z), may also be modified. Accordingly, the digital system may be used to generate digital orthotic insoles from the scanned image.

A medical professional may also be able to modify through the digital system the height of wedges of the hindfoot and forefoot of the digital orthotic insoles. The digital system may also provide a way of modifying and/or correcting the medial longitudinal arc and/or the lateral longitudinal arch.

Finally, the digital system may have means of transferring the information relating to the personalized digital orthotic insole to a printing system e.g. a 3D printing system, such that the designed orthotic insole is 3D printed.

Although only a number of examples have been disclosed herein, other alternatives, modifications, uses and/or equivalents thereof are possible. Furthermore, all possible combinations of the described examples are also covered. Thus, the scope of the present disclosure should not be limited by particular examples, but should be determined only by a fair reading of the claims that follow.

1. A scanning device for imaging a human extremity comprising:
a transparent base comprising a first side and a second side, the first side being configured to accommodate the human extremity;
a stereoscopic camera comprising one or more infrared lasers, configured to emit a laser beam which hits the second side of the transparent base in one or more incidence points, and
wherein one or more opaque items are located between the one or more laser beams and the one or more incidence points.

2. The scanning device of claim 1, wherein the stereoscopic camera is a static camera.

3. The scanning device of claim 1, wherein the human extremity is a human foot.

4. The scanning device of claim 3, wherein the transparent base is configured to accommodate a sole of the human foot.

5. The scanning device of claim 1, wherein the transparent base is made of methacrylate or glass.

6. The scanning device of claim 1, wherein the second side of the transparent base is an inner side of the scanning device

7. The scanning device of claim 1, wherein the opaque items are arranged on or in the transparent base, and wherein the number of opaque items on or in the transparent base is equal to the number of infrared lasers comprised in the stereoscopic camera.

8. The scanning device of claim 1, wherein the stereoscopic camera comprises at least two lenses, and wherein the camera comprises one infrared laser per lens.

9. The scanning device of claim 1, wherein the opaque items cover at least an area of the incidence points of the laser beams.

10. The scanning device of claim 1, wherein the opaque items are attached to the second side of the transparent base.

11. The scanning device of claim 1, wherein the opaque items are stickers.

12. The scanning device of claim 1, wherein the opaque items comprise a round or squared shape.

13. A method for imaging a human extremity comprising:
providing an imaging device for imaging the human extremity comprising
a transparent base comprising a first side and a second side, the first side being configured to accommodate the human extremity;
a stereoscopic camera comprising one or more infrared lasers, configured to emit a laser beam which hits the second side of the transparent base in one or more incidence points, and
wherein one or more opaque items are located between the one or more laser beams and the one or more incidence points; and
the method further comprising:
accommodating the human extremity on the first side of the transparent base;
scanning and obtaining a 3D image.

14. The method of claim 13, wherein processing the 3D image comprises removing the opaque items from the obtained image.

15. The method of claim 13, further comprising processing the 3D image and obtaining a 3D image comprising only the accommodated human extremity.

16. A scanning device for imaging a human extremity comprising:
a transparent base comprising a first side and a second side, the first side being configured to accommodate the human extremity;
a stereoscopic camera comprising one or more infrared lasers, configured to emit a laser beam which hits the second side of the transparent base in one or more incidence points, and
wherein one or more opaque stickers are arranged on the transparent base between the one or more laser beams and the one or more incidence points.

17. The scanning device of claim 16, wherein the stereoscopic camera is a static camera.

18. The scanning device of claim 16, wherein the human extremity is a human foot.

19. The scanning device of claim 16, wherein the one or more opaque stickers are attached to the second side of the transparent base.

20. The scanning device of claim 16, wherein the stereoscopic camera comprises at least two lenses, and wherein the camera comprises one infrared laser per lens.
